(19) United States
(12) Patent Application Publication
(10) Pub. No.: US 2008/0171971 A1
(43) Pub. Date: Jul. 17, 2008

(54) Novel enhanced device and technique for mixing and dispensing a preserved agent

(76) Inventors: Paul Mario DiPerna, San Clemente, CA (US); Steven Donald Kimmell, Granada Hills, CA (US); Jodi Laurel McAlpine, Simi Valley, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 11/274,997

(22) Filed: Nov. 16, 2005

(51) Int. Cl. A61M 37/00

(52) U.S. Cl. 604/82

(57) ABSTRACT

A new and enhanced device and technique for mixing and dispensing a preserved agent is disclosed. The syringe-like device employs a novel piston fluid channel allowing for various chambers of the syringe to come into fluid communication when the piston is actuated. More especially, the person administering the preserved agent may visually confirm full resuspension and deliver an accurate dosage of the agent.

### BACKGROUND

The present invention relates to the mixing and dispensing of sensitive biological and bioactive materials, including, for example, preserved agents. More particularly, the invention concerns a device for the admixing of a powdered or otherwise preserved agent, such as a lyophilized drug, and a fluid through a specialized fluid channel in the piston of a syringe. This invention allows lyophilized agents such as Botox, Factor VIII, Immunoglobulin IgG, and many others to be delivered. Moreover these drugs can be stored and utilized without the need for syringes, complicated sterile technique, or professional training in the mixing of drugs in remote places such as on the battlefield or in rural areas.

State of the art information relevant to attempts to address the problem of resuspension of a preserved agent prior to its delivery can be found in U.S. Pat. Nos. 4,941,876; 5,330,426; 5,531,683; 5,637,087; 5,785,682; and 6,387,074, as well as U.S. Publication Nos. 2004/0138611 and 2005/0075602, each of which is expressly incorporated by reference as if fully set forth herein.

However, each of these references suffer from one or more of the following disadvantages: incomplete admixture of the fluid and the preserved agent, inaccurate dosage delivery of the resuspended agent, labor intensive devices, multiple sterile systems that increase the potential for compromises of sterile technique, and the inability to fully complete the admixing process in front of the patient. These various limitations make the present disclosure more desirable than the cited references, and are demonstrative of satisfaction of a longstanding need in the art addressed and overcome according to the teachings of the present disclosure.

### SUMMARY

The present disclosure serves as a simple solution to the myriad of formerly complicated solutions for the resuspension and delivery of preserved agents. The present system allows for simple loading and administration of preserved agents after resuspension in an appropriate fluid, which occurs completely within the device. This type of system is advantageous because it saves labor and reduces the probability of error inherent in multiple step processes involving systems with multiple needles, wipes for bottle tops, and other mistakes that potentially compromise sterility of the instrument and agent vehicle. Those skilled in the art will understand that any biological industrial product can be used with and is embraced by the instant disclosure.

According to a feature of the present disclosure, a piston fluid channel is used to admix a fluid with a preserved agent prior to delivery of the agent to a patient. A preserved agent ampoule in a first position holds the preserved agent. The ends of the preserved agent ampoule are pierceable septa. Fluid is loaded into a fluid chamber in the dispenser body prior to placing the piston into the dispenser body.

As force is applied to the piston via its plunger, a cannula on the end of the piston pierces the septum on the piston end of the preserved agent ampoule. The fluid is moved from a fluid chamber into a preserved agent ampoule chamber through a piston fluid channel and the piston cannula. A combination of at least one of compression force, a partial vacuum in the preserved agent ampoule chamber, or some combination of both causes the fluid to move through the piston fluid channel into the preserved agent ampoule chamber.

Once in the preserved agent ampoule chamber, the fluid and the preserved agent admix to form a resuspended agent ready for delivery to a patient. The person operating an embodiment of the present disclosure can, at this point, visually confirm that the preserved agent has been fully resuspended in the fluid prior to final delivery.

After resuspension of the preserved agent, additional force is applied to the piston by means of the plunger. This additional force moves the entire preserved agent ampoule into a second position, towards the interior end of the delivery cannula, which pierces the delivery end septum of the preserved agent ampoule and places the delivery cannula into the preserved agent ampoule chamber. In this position, the delivery cannula provides a channel to deliver the resuspended agent to the patient.

Additional force applied to the piston moves the piston end septum of the preserved agent ampoule through the preserved agent ampoule towards the delivery end septum. The compression force within the preserved agent ampoule chamber forces the resuspended agent into the delivery cannula and into the patient.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1 is a longitudinal sectional view of an embodiment of the present disclosure showing a piston and preserved agent ampoule in their initial configuration prior to piercing of either a piston end or a delivery end septum.

FIG. 2 is a longitudinal sectional view of an embodiment of the present disclosure during a piercing operation of a piston end septum by a piston cannula.

FIG. 3 is a longitudinal sectional view of an embodiment of the present disclosure of a piston during an admixing process of a fluid and a preserved agent in a preserved agent ampoule chamber.

FIG. 4 is a longitudinal sectional view of an embodiment of the present disclosure of the process with a piston moving a piston end septum towards a delivery end septum and delivering a resuspended agent through a delivery cannula.

FIG. 5 is a longitudinal sectional view of an embodiment of the present disclosure wherein the piston cannula is a spike, which forms one part of a single-piece molded piston and cannula unit.

FIG. 6 is a longitudinal sectional view of an embodiment with an adjustable cannula assembly allowing for variations in the volume of fluid delivered into a preserved agent ampoule chamber.

FIG. 7 is a longitudinal sectional view of an embodiment with an adjustable cannula assembly allowing for variations in the volume of fluid delivered into a preserved agent ampoule chamber.

FIG. 8 is a longitudinal sectional view of an embodiment of the process of adjusting an adjustable cannula assembly allowing for variations in the volume of fluid delivered into a preserved agent ampoule chamber.

### DETAILED DESCRIPTION

The present inventors discovered that certain biological, chemical, and pharmaceutical agents and compounds can be delivered via a novel piston fluid channel mechanism. According to embodiments of the present disclosure, piston fluid channel 121 allows for complete admixture of fluid 129 and preserved agent 107 within the container originally holding preserved agent 107. Once preserved agent 107 is admixed with fluid 129, resuspended agent 133 can then be delivered.

Likewise disclosed is a novel enhanced syringe-style device wherein the instant disclosure describes a novel method for mixing and dispensing preserved agent 107.

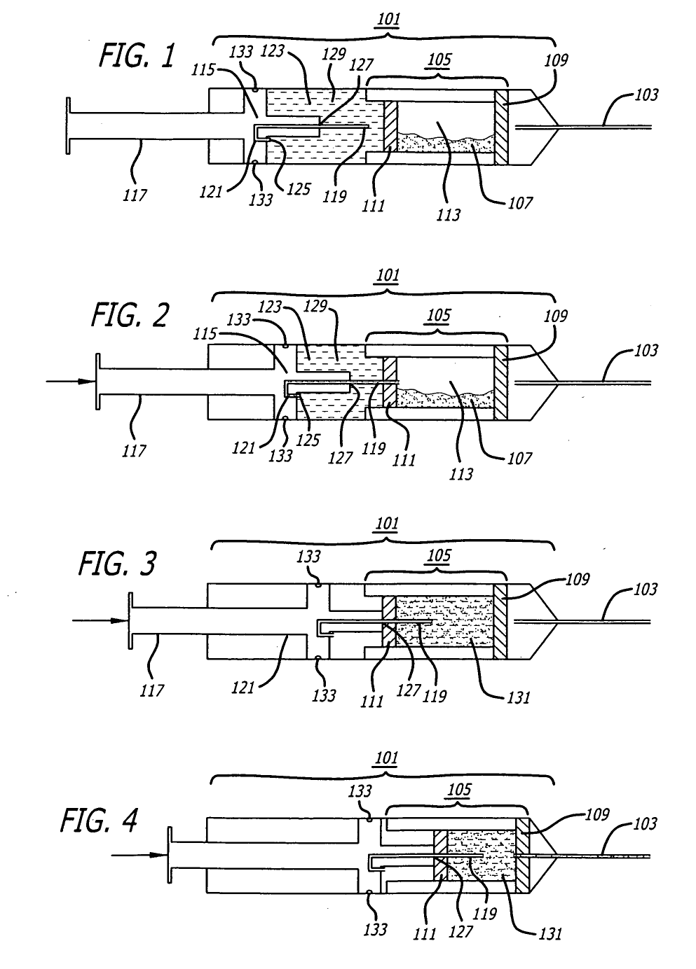

FIG. 1 illustrates embodiments of aspects of an embodiment of the present disclosure in an initial configuration. The system comprises dispenser body 101 into which preserved agent ampoule 105 is inserted prior to insertion of fluid 129 and piston 115. Piston 115 contains piston fluid channel 121 and piston cannula 119, which together move fluid 129 from fluid chamber 123 into preserved agent ampoule chamber 113 for admixing of fluid 129 with preserved agent 107. Thereafter, piston 115 provides the force by which resuspended agent 131 is delivered through delivery cannula 103.

Dispenser body 101 is an open-ended container with a delivery end and a piston end according to an embodiment of the present disclosure. Delivery end of dispenser body 101 narrows to an articulation point where delivery cannula 103 connects for delivery of resuspended agent 131. Delivery cannula 103 can be a hypodermic needle or any other suitable dispensing structure as commonly known in the art. The interior end of delivery cannula 103 is either a part of cannula itself or a secondary structure, such as a spike, in fluid communication with delivery cannula 103 and providing a sealed channel in which resuspended agent 131 can be delivered. The interior end of delivery cannula 103 protrudes into the cavity of dispenser body 101 and pierces delivery end septum 109 of preserved agent ampoule 105 preparatory to delivery of resuspended agent 131.

According further to the illustrative example, preserved agent ampoule 105 is a hollow container with a delivery end and a piston end. Preserved agent ampoule 105 contains preserved agent 107. The delivery end is sealed with delivery end septum 109, which is made of a flexible material suitable to piercing by delivery cannula 103 as commonly known in the art. Delivery end septum 109 is fixed with respect to its position in preserved agent ampoule 105.

Preserved agent ampoule 105 contains preserved agent 107 and is inserted into dispenser body 101 through the opening in the piston end of dispenser body 101. According to one version of the instant teachings, once inserted into dispenser body 101, preserved agent ampoule 105 abuts a positioning protuberance, which are common in the art, contained within the inside surface of the delivery end of dispenser body 101. The protuberance holds preserved agent ampoule 105 in a first position for the piercing of piston end septum 111 with piston cannula 119.

A sufficient force moves preserved agent ampoule 105 past the positioning protuberance and into a second position. Thus, the positioning protuberance gives sufficient friction to prevent movement of preserved agent ampoule 105 during piercing of piston end septum 111, but not so much friction that additional force cannot move preserved agent ampoule 105. The positioning protuberance can be made of any suitable material capable of holding preserved agent ampoule 105 in place when a weaker but sufficient force is exerted on preserved agent ampoule 105 allowing piston cannula 119 to pierce piston end septum 111, but also permitting preserved agent ampoule 105 to move into a second position when a stronger force is exerted on it. The protuberance, in an exemplary embodiment, is a bearing-like member as commonly known in the art. Another embodiment is a positioning member, which holds preserved agent ampoule 105 in the first position. Positioning member extends from the inside to the outside of dispenser body 101. After piston cannula 119 pierces piston end septum 111 and fluid 129 is moved into preserved agent ampoule chamber 113, positioning member is removed and additional force exerted on preserved agent ampoule 105 moves it into the second position.

According to exemplary embodiments, piston end septum 111 is made of a material suitable to form a tight seal with the inner walls of preserved agent ampoule 105, but is also moveable within preserved agent ampoule 105 while maintaining the seal. The material is also suitable for piercing by piston cannula 119 as commonly known in the art. Piston end septum 111 should be made of a flexible material, such as rubber, so that the volume of fluid chamber 123 will not change during the piercing process. Alternately, a small pocket of air in fluid chamber 123 accomplishes the same objective.

In an embodiment, piston end septum 111 is a two piece system comprised of a support piece that moves through preserved agent ampoule chamber 113 and a pierceable region, similar to bottle tops commonly known in the art. The support piece can be made of metal, plastic, or other materials suitable for being pushed through preserved agent ampoule chamber 113. The support piece either forms a seal with the inner wall or walls of preserved agent ampoule 105 or has a separate sealing member connected. The pierceable region is made as previously described for piston end septum 111. Moreover, the region must be large enough for piston cannula 119 to reliably pierce it.

Piston end septum 111 forms a seal with preserved agent ampoule 105 via friction between piston end septum 111 and inner wall or walls of preserved agent ampoule 105. Unlike delivery end septum 109, piston end septum 111 is movable with respect to preserved agent ampoule 105. Applied force to piston end septum 111 will move it through preserved agent ampoule chamber 113 towards delivery end septum 109 in the delivery process of resuspended agent 131.

In an illustrative embodiment, preserved agent ampoule 105 is sealed under vacuum conditions. The vacuum is intended to move fluid 129 from fluid chamber 123 to preserved agent ampoule chamber 113, through piston fluid channel 121 and piston cannula 119, in the absence of compression force. However, vacuum conditions are not necessary; fluid 129 can be moved from fluid chamber 123 into preserved agent ampoule 105 via compression of fluid chamber 123 by movement of piston 115 toward the delivery end of dispenser body 101, which raises the internal pressure of fluid chamber 123, consequently forcing fluid 129 through piston fluid channel 121 and piston cannula 119, and into preserved agent ampoule chamber 113 as previously described.

In an embodiment, dispenser body 101 and preserved agent ampoule 105 are made of a transparent material suitable to visual inspection of the mixing process. The person administering the agent can visually verify resuspension of preserved agent 107 into fluid 129, ensuring that the complete dosage of preserved agent 107 resuspends into fluid 129. Additionally, the entire process can be performed in front of the patient.

Referring still to an illustrative embodiment in FIG. 1, piston 115 completes the assembly of the present disclosure as shown in an initial configuration. Plunger 117 connects to piston 115 and is actuated to apply force to piston 115 and move it through dispenser body 101. Plunger 117 can be made of any material suitable for syringe plungers.

When inserted into dispenser body 101, at least one sealing member 133 of piston 115 articulates with the inner wall or walls of dispenser body 101 to form a seal. Each sealing member 133 is made of a suitable material, such as rubber, plastic, polymers, ePTFE, and the like, allowing for a tight seal between sealing member 133 and the inner wall or walls of dispenser body 101. Each sealing member 133 also allows for movement of piston 115 when axial force is applied to piston 115. Such movement does not break the seal between each sealing member 133 and the inner wall or walls of dispenser body 101.

Piston 115 also contains piston fluid channel 121. Piston fluid channel opening 125 and piston cannula opening 127 are the end points of piston fluid channel 121. Piston fluid channel 121 forms a sealed and continuous conduit between fluid chamber 123 and preserved agent ampoule chamber 113. Thus, piston fluid channel 121 allows fluid 129 to flow from fluid chamber 123 to preserved agent ampoule chamber 113 when vacuum force or compressive force is applied to fluid chamber 123. In an embodiment, piston fluid channel opening 125 is placed in a location where the entire volume of fluid 129 may be removed from fluid chamber 123 and moved into preserved agent ampoule chamber 113.

Piston cannula 119 extends from piston 115 and is used to pierce piston end septum 111. In an embodiment, the delivery end of piston cannula 119 is a trocar suitable for piercing piston end septum 111. Piston cannula 119 can be made from any suitable material commonly known in the art to be used for piercing, such as hypodermic needles or spikes. Piston cannula 119 also serves to move fluid 129 from fluid chamber 123 to preserved agent ampoule chamber 113. Thus, it can be considered an integral part of the fluid transport system of the present disclosure. In an embodiment, piston cannula 119 and the interior end of delivery cannula 103 are off-axis of each other, which prevents piston cannula 119 from abutting into delivery cannula 103 in the process of delivering resuspended agent 131.

According still to an illustrative embodiment of FIG. 1, fluid chamber 123 is defined by dispenser body 101 on the sides, preserved agent ampoule 105 on the delivery end, and piston 115 on the piston end. Both piston 115 and preserved agent ampoule 105 seal fluid chamber from leakage, effectively forcing fluid 129 to move through piston fluid channel 121 when compressive or vacuum force is applied to the chamber. In another embodiment, a sealing membrane can be used to form the delivery end of fluid chamber 123. The membrane abuts with piston end septum 111 so that piercing the membrane and piston end septum 111 occur in close succession or simultaneously to prevent the loss of fluid 129 in the space between fluid chamber 123 and preserved agent ampoule 105.

Referring again to FIG. 1, a mixing and dispensing apparatus according to an embodiment of the present disclosure operates as follows. First, a preserved agent 107 is loaded into preserved agent ampoule 105. In an embodiment, creation of preserved agent 107 can also occur in preserved agent ampoule 105 prior to being sealed by piston end septum 111. After loading preserved agent 107, piston end septum 111 is applied to preserved agent ampoule 105.

Delivery of resuspended agent 131 by compression force within preserved agent ampoule chamber 113 allows a precise and accurate delivery of a predetermined dosage of resuspended drug 131 to the patient. Moreover, if the factory preloads the system, the entire apparatus can be opened and operated in the presence of the patient, which allays patient concerns about safety and contamination stemming from improper sterile technique or reuse of hypodermic needles.

In another embodiment, the system is flexible. Dispenser body 101 and piston 115 are reusable after being properly cleaned and sterilized. Additionally, doctors can choose from various available fluids when fluid 129 is loaded on-site. Reuseable core components provide a mixing and dispensing apparatus that is both cost effective and simple to load and operate. Additionally, it also gives doctors greater flexibility in the way preserved agent 107 is mixed, in addition to the concentration of the dose administered. These features make embodiments of the present disclosure superior to other devices on the market today.

An embodiment of the present disclosure also includes a backflow restrictor built into piston cannula 119 or piston fluid channel 121, as is commonly known in the art. The backflow restrictor allows fluid 129 to flow unidirectionally from piston fluid channel opening 125 towards piston cannula opening 127. One such embodiment comprises a one-way valve, as commonly known to artisans. In its closed state, the one-way valve is positioned such that the edges form a tight seal within piston fluid channel 121 or piston cannula 119. When fluid 129 flows in the proper direction from piston fluid channel opening 125 towards piston cannula opening 127, the one-way valve is forced into an open position by the flow of the fluid. However, fluid flowing from piston cannula opening 127 to piston fluid channel opening 125 forces the one-way valve to close, which seals piston fluid channel 121 to back flow.

Consequently, as piston end septum 111 moves through preserved agent ampoule chamber 113 toward delivery end septum 109, the pressure in preserved agent ampoule chamber 113 increases, which seals piston fluid channel 121 and forces resuspended agent 131 to flow into delivery cannula 103, rather than back through piston cannula 119 and fluid channel 121 into fluid chamber 123.

Another embodiment includes, in addition to that already disclosed, a lever that, when activated, advances preserved agent ampoule 105 into a second position as shown in FIG. 4. The lever rests on preserved agent ampoule 105 and contains an activator positioned on the exterior of dispenser body 101. When activated, the portion of the lever resting on preserved agent ampoule 105 exerts sufficient force push preserved agent ampoule 105 into the second position preparatory to delivery of resuspended agent 131.

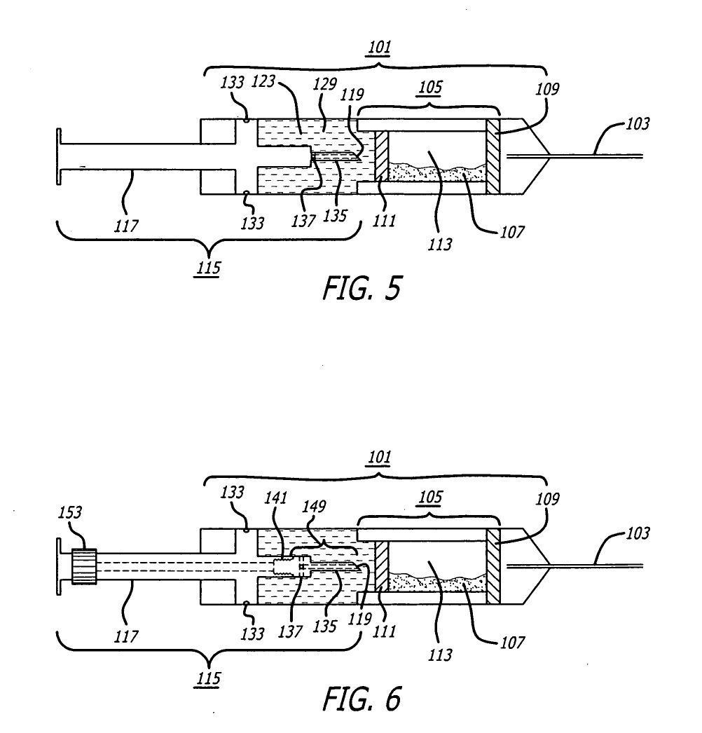

An embodiment of the present disclosure as illustrated in FIG. 5 comprises a single-piece piston assembly. As shown in FIG. 5, the piston cannula 119 is spike port 135. At least one opening forms piston fluid channel opening 125. The end of spike port 135 forms piston cannula opening 127. The mechanism of delivery of fluid 129 into preserved agent ampoule chamber 113 occurs as previously described.

Spike ports are well known in the art and easy to manufacture. Spike port 135 can be integrated into piston 115 in a single molding process. Thereafter, at least one opening is created forming piston fluid channel opening 125, which is created so as to be in fluid communication with piston cannula opening 127. Thus, piston fluid channel opening 125, piston fluid channel 121, and piston cannula opening 127 form a sealed, continuous channel by which fluid chamber 123 is put into fluid communication with preserved agent ampoule chamber 113. Spike port 135 can be made of materials that are well known in the art.

In an illustrative embodiment as shown in FIG. 6, piston 115 further contains adjustment mechanism 141 connected to fluid adjustment actuator 153 on plunger 117. Turning fluid adjustment actuator 153 advances or retracts cannula assembly 149, which comprises at least piston fluid channel 121 and piston cannula 119. Adjustment of cannula assembly 149 allows for variations in the volume of fluid 129 to be moved into preserved agent ampoule chamber 113, allowing for the preparation of various concentrations of resuspended agent 131.

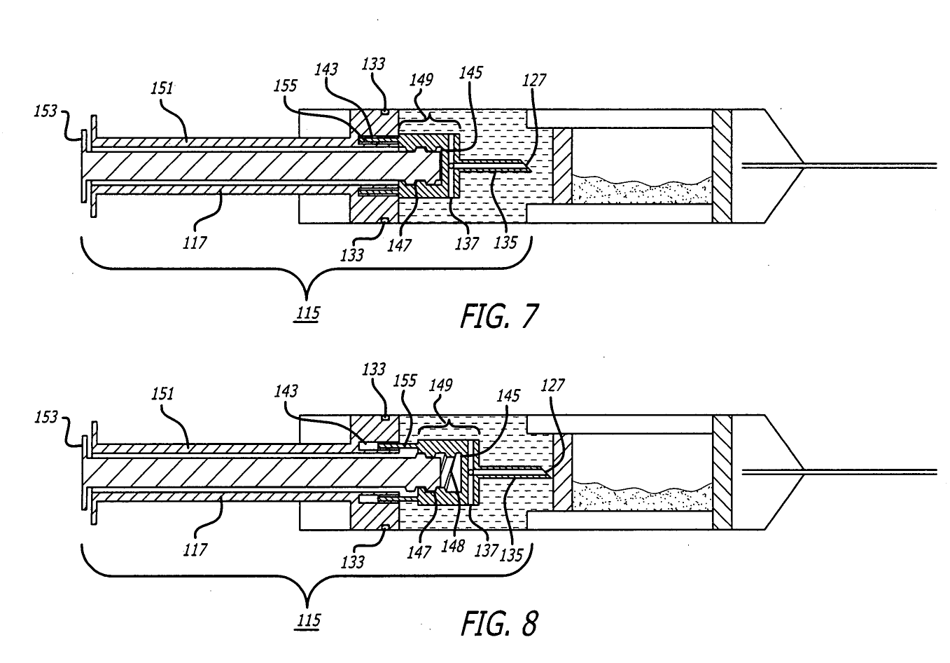

An illustrative example of similar adjustment mechanism 141 comprises a screw-like mechanism allowing the axial length of cannula assembly 149 to be adjusted. As seen in greater detail in an illustrative example in FIGS. 7 and 8, fluid adjustment actuator 153 is turned to actuate the screw-like mechanism that advances or retracts cannula assembly 149. Specifically, plunger housing 151 houses a threaded fluid adjustment actuator 153. Fluid adjustment actuator 153 has a delivery end, which has plunger threads 147. Plunger threads 147 articulate with cannula assembly threads 148 such that when fluid adjustment actuator 153 is turned, cannula assembly advances or retracts axially along the long axis of the present disclosure.

Cannula assembly 149 contains at least one cannula assembly positioning member 155 to ensure that piston 115 and cannula assembly 149 remain correctly aligned relative to each other. Within the piston, at least one cannula assembly receiver 143 receives cannula assembly positioning member 155 as shown in FIGS. 7 and 8. Each cannula assembly receiver 143 receives at least one cannula positioning member 155.

In a similar embodiment, the cannula assembly 149 adjustment mechanism comprises a notched positioning mechanism allowing the axial length of cannula assembly 149 to be adjusted. Fluid adjustment actuator 153 is connected to the cannula assembly. The cannula assembly contains a protuberance that articulates with notches in piston 121. In a first position, the protuberance fits into one of a plurality of notches, fixing the position of the protuberance with respect to the notch. Rotating the cannula assembly 149 moves it into a second position where cannula assembly 149 is not positioned in a notch and may move axially. The plurality of notches allows for a plurality of fixed positions for cannula assembly 149 with respect to piston 115. Thus, variations in the volume of fluid 129 moved into preserved agent ampoule chamber 113 is accomplished.

Referring to an illustrative method, preserved agent ampoule 105 is loaded into dispenser body 101 as shown in FIG. 1 either at the factory or at the site of delivery of the agent. After preserved agent ampoule 105 is loaded, fluid 129 is placed into dispenser body 101. Thereafter, piston 115 is inserted into dispenser body 101 on the piston end, thereby forming fluid chamber 123 between piston 115, the dispenser body 101, and preserved agent ampoule 105. This combination of steps forms the configuration depicted in FIG. 1. The loading of fluid 129 into fluid chamber 123 and insertion of piston 115 into dispenser body 101 are also conducted under sterile conditions.

According to an embodiment of an illustrative method, sealing of preserved agent ampoule 105 may occur under vacuum conditions sufficient to move fluid 129 from fluid chamber 123 into preserved agent ampoule 105 when piston end septum 111 is pierced. The vacuum pressure should not be so great as to prematurely dislodge piston end septum 111 towards delivery end septum 109. The process of loading preserved agent 107 into preserved agent ampoule chamber 113 should be conducted under sterile conditions.

FIG. 2 shows an embodiment of the process of resuspension of preserved agent 107 in fluid 129, which occurs in preserved agent ampoule 105. In order to move fluid 129 into preserved agent ampoule 105, dispenser body 101 is held in an inverted position such that the force of gravity holds fluid 129 against piston 115 and over piston fluid channel opening 125. Force is applied to piston 115 driving it into dispenser body 101 towards the delivery end. The net result forces piston cannula 119 to pierce piston end septum 111 such that piston cannula opening 127 is placed into preserved agent ampoule chamber 113, which puts fluid chamber 123 in fluid communication with preserved agent ampoule chamber 113, as shown in FIG. 2. The application of either continued compression force on piston 115 or a vacuum in preserved agent ampoule chamber 113 or a combination of these forces moves fluid 129 from fluid chamber 123 into preserved agent ampoule chamber 113 through piston fluid channel 121.

Referring now to an embodiment represented in FIG. 3, mixture of fluid 129 with preserved agent 107 in preserved agent ampoule chamber 113 resuspends preserved agent 107. Resuspended agent 131 is delivered via delivery cannula 103. In one embodiment, preserved agent ampoule 105 and dispenser body 101 are transparent, allowing the person administering resuspended agent solution to visually confirm that preserved agent 107 is fully admixed with fluid 129 prior to delivery of resuspended agent 131. This extra step increases the accuracy of the dosage concentration delivered to each patient.

According still to FIG. 3, force applied to piston 115 drives piston 115 to a position where it rests against preserved agent ampoule 105. Continued pressure on piston 115 moves preserved agent ampoule 105 into a second position preparatory for delivery of resuspended agent 131. As preserved agent ampoule 105 moves towards its second position, the interior end of delivery cannula 103 pierces delivery end septum 109 of preserved agent ampoule 105 such that delivery cannula 103 is placed into fluid communication with preserved agent ampoule chamber 113 as shown in FIG. 4.

According to an embodiment demonstrated in FIG. 4, additional pressure applied to piston 115 drives piston end septum 111 towards delivery end septum 109. Once the interior end of delivery cannula 103 pierces delivery end septum 109, preserved agent ampoule 105 ceases to move with continued pressure on piston 115. However, as additional force is applied to piston 115, piston end septum 111 moves through preserved agent ampoule chamber 113 towards delivery end septum 109, which reduces the volume of preserved agent ampoule chamber 113. Delivery of resuspended agent 131 is thus accomplished by the reduction in volume of preserved agent ampoule chamber 113. This forces resuspended agent 131 into the interior end of delivery cannula 103, through delivery cannula 103, and out the exterior end of delivery cannula 103, thereby effectuating delivery of resuspended agent 131.

An embodiment of a method of delivering a specified volume of fluid 129 into preserved agent ampoule chamber 113 is demonstrated in FIGS. 7 and 8. As shown in FIG. 7, cannula assembly is in a first position. Turning fluid adjustment actuator 153 advances or retracts cannula assembly 149 as shown in FIG. 8. During the adjustment process, the articulation of cannula assembly positioning member 143 and cannula assembly receiver 155 preserve the correct alignment of cannula assembly 149 relative to piston 115. Gradation marks may be used to allow precise delivery of a specified volume of fluid 129 into preserved agent ampoule chamber 113.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

What is claimed is:

1. A preserved agent delivery apparatus for mixing and resuspending a preserved agent comprising, in combination:
(a) a syringe-like body;
(b) a preserved agent ampoule;
(c) a piston, wherein the piston is further comprised of:
i) a piston actuator;
ii) a piston cannula; and
iii) a piston sealing member, whereby a seal with at least one inner wall of the syringe like body is maintained; and
(d) a piston fluid channel, wherein the piston fluid channel is further comprised of:
i) a piston fluid channel opening;
ii) a piston cannula opening; and
iii) a channel, wherein the endpoints of the channel are the piston fluid channel opening and the piston cannula opening;
wherein the piston cannula has a piston end and a delivery end, and the piston end connects to the piston cannula opening such that fluid passes from the piston fluid channel into the piston cannula without appreciable loss of a fluid;
wherein the fluid is moved through the piston fluid channel from a fluid chamber into a preserved agent ampoule chamber.

2. The apparatus of claim 1, wherein the preserved agent ampoule is further comprised of:
(a) at least one side wall;
(b) a delivery end septum, wherein the delivery end septum is sealed with respect to the at least one side wall of the preserved agent ampoule and may be pierced with a delivery cannula while retaining a seal around the delivery cannula;
(c) a piston end septum, wherein the piston end septum forms a seal with the at least one side wall and may move with respect to the at least one side wall while retaining a seal with the at least one side wall; and
may be pierced with the piston cannula while retaining a seal around the piston cannula;
(d) the preserved agent ampoule chamber formed from the articulation of the at least one side wall, delivery end septum, and piston end septum; and
(e) a preserved agent.

3. The apparatus of claim 1, wherein the system is used to deliver a preserved agent comprising a toxin of Clostridium botulinum.

4. The apparatus of claim 1, further comprising a piston fluid channel sealing member that:
(a) rests in a first position when the fluid flows from the piston fluid channel opening towards the piston cannula opening; and
(b) rests in a second position when the fluid flows from the piston cannula opening towards the piston fluid channel opening;
wherein the piston fluid channel sealing member allows free flow of the fluid when in the first position, but seals the piston fluid channel when in the second position; and
wherein the piston fluid channel sealing member adjusts its position based on one of fluid pressure, chamber pressure, and a combination of both.

5. The apparatus of claim 1, wherein the piston fluid channel opening is placed in a position where substantially all of the fluid in the fluid chamber may be removed.

6. The apparatus of claim 1, wherein the length measured from the piston sealing member to one of:
a first point of contact between the piston and the preserved agent ampoule that prevents further ingress of the piston cannula into the preserved agent ampoule chamber; and
the delivery end of the piston cannula;
is adjustable.

7. The apparatus of claim I wherein the piston is further comprised of:
(a) a cannula assembly comprised of the piston fluid channel, the piston cannula, and a first adjustment member;
(b) a second adjustment member; and
(c) a fluid adjustment actuator;
wherein the fluid adjustment actuator connects to the second adjustment member and the second adjustment member may be actuated with the fluid adjustment actuator;
wherein the first adjustment member and the second adjustment member articulate such that actuation of the second adjustment member simultaneously actuates the first adjuster;
wherein actuation of the first adjustment member results in axially advancing or retracting the cannula assembly relative to the piston.

8. An apparatus for mixing and delivering a preserved agent comprising:
(a) a syringe-like body;
(b) a delivery cannula;
(c) a preserved agent ampoule, wherein the preserved agent ampoule is further comprised of:
i) a delivery end septum;
ii) a piston end septum,
iii) at least one side wall;
iv) a preserved agent ampoule chamber; and
v) a preserved agent;
(d) a piston, wherein the piston is further comprised of:
i) a piston fluid channel;
ii) a piston sealing member; and
iii) a piston cannula;
wherein the piston fluid channel and the piston cannula are in fluid communication;
wherein the piston fluid channel is defined to be a sealed and continuous channel between at least one piston fluid channel opening and at least one piston cannula opening; and
(e) a fluid chamber in fluid communication with at least one piston fluid channel opening;
wherein actuation of the piston causes the piston cannula to be placed into the preserved agent ampoule chamber, whereby the fluid chamber and the preserved agent ampoule chamber are brought into fluid communication;
wherein additional actuation of the piston forces a fluid in the fluid chamber to be moved into the preserved agent ampoule chamber allowing for admixture of the fluid and the preserved agent, whereby a resuspended agent is formed; and
wherein further actuation of the piston places the delivery cannula into fluid communication with the preserved agent ampoule chamber;
wherein further actuation of the piston delivers the resuspended agent through the delivery cannula by moving the piston end septum through the preserved agent ampoule chamber.

9. The apparatus of claim 8, further comprising a piston fluid channel sealing member that:
(a) rests in a first position when the fluid flows from the at least one piston fluid channel opening towards the at least one piston cannula opening; and
(b) rests in a second position when the fluid flows from the at least one piston cannula opening towards the at least one piston fluid channel opening;
wherein the piston fluid channel sealing member allows free flow of the fluid when in the first position, but seals the piston fluid channel when in the second position; and
wherein the piston fluid channel sealing member adjusts its position based on one of fluid pressure, chamber pressure, and a combination of both.

10. The apparatus of claim 8, wherein the length measured from the piston sealing member to one of:
a first point of contact between the piston and the preserved agent ampoule that prevents further ingress of the piston cannula into the preserved agent ampoule chamber; and
a delivery end of the piston cannula;
is adjustable.

11. The apparatus of claim 8 wherein the piston is further comprised of:
(a) a cannula assembly comprised of the piston fluid channel, the piston cannula, and a first adjustment member;
(b) a second adjustment member; and
(c) a fluid adjustment actuator;
wherein the fluid adjustment actuator connects to the second adjustment member and the second adjustment member may be actuated with the fluid adjustment actuator;
wherein the first adjustment member and the second adjustment member articulate such that actuation of the second adjustment member simultaneously actuates the first adjustment member;
wherein actuation of the first adjustment member results in axially advancing or retracting the cannula assembly relative to the piston.

12. A method of mixing and delivering a preserved agent comprising the steps of:
(a) admixing a preserved agent and a fluid, wherein the fluid is held in a fluid chamber and moved into contact with the preserved agent through a piston fluid channel, wherein the movement of the fluid through the piston fluid channel comprises the following steps:
i) moving the fluid through a piston fluid channel opening;
ii) moving it through the piston fluid channel; and
iii) moving out of a piston cannula opening.
(b) admixing the fluid and the preserved agent to form a resuspended agent; and
(c) delivering the resuspended agent.

13. The method of claim 12, further comprising the steps of:
(a) moving the fluid from the piston cannula opening into a piston cannula with no appreciable loss of the fluid; and
(b) delivering the fluid from the piston cannula into a preserved agent ampoule chamber holding the preserved agent.

14. The method of claim 13, further comprising the additional steps of:
(a) piercing a piston end septum of a preserved agent ampoule with the piston cannula by applying force to a piston further comprised by the piston cannula;
(b) piercing a delivery end septum of the preserved agent ampoule with a delivery cannula wherein the delivery cannula is placed into fluid communication with the preserved agent ampoule chamber holding the resuspended agent; and
(c) moving the piston end septum through the preserved agent ampoule chamber towards the delivery end septum wherein the piston end septum maintains a seal with at least one inner wall of the preserved agent ampoule, thereby delivering the resuspended agent from the preserved agent ampoule chamber through the delivery cannula;
wherein the steps are in no particular order.

15. The method of claim 12, comprising the additional step of varying the length measured from a piston sealing member to one of:
(a) a first point of contact between a piston and a preserved agent ampoule that prevents further ingress of a piston cannula into a preserved agent ampoule chamber; and
(b) a delivery end of the piston cannula;
whereby various volumes of the fluid may be moved through the piston fluid channel causing variations in the concentration of the resuspended agent.

16. The method of claim 12 further comprising the step of varying the volume of the fluid moved into a preserved agent ampoule chamber using:
(a) a cannula assembly comprised of the piston fluid channel, a piston cannula, and a first adjustment member;
(b) a second adjustment member; and
(c) a fluid adjustment actuator;
wherein the fluid adjustment actuator connects to the second adjustment member and the second adjustment member may be actuated with the fluid adjustment actuator;
wherein the first adjustment member and the second adjustment member articulate such that actuation of the second adjustment member simultaneously actuates the first adjustment member;
wherein actuation of the first adjustment member results in axially advancing or retracting the cannula assembly relative to a piston.

17. The method of claim 12, where in the preserved agent is a lyophilized drug.

18. The method of claim 12, wherein the preserved agent delivered comprises a toxin of Clostridium botulinum.

19. The method of claim 12, wherein the fluid is moved by at least one of the group containing compression force, vacuum force, and a combination of compression force and vacuum force.

20. The method of claim 12, further comprising the step of preventing back flow through the piston fluid channel using a piston fluid channel sealing member that:
(a) rests in a first position when the fluid flows from the piston fluid channel opening towards the piston cannula opening; and
(b) rests in a second position when the fluid flows from the piston cannula opening towards the piston fluid channel opening;
wherein the piston fluid channel sealing member allows free flow of the fluid when in the first position, but seals the piston fluid channel when in the second position; and
wherein the piston fluid channel sealing member adjusts its position based on one of fluid pressure, chamber pressure, and a combination of both.
